(19) United States
(12) Patent Application Publication
(10) Pub. No.: US 2012/0142094 A1
(43) Pub. Date: Jun. 7, 2012

(54) GENERATING IPS CELLS BY PROTEIN TRANSDUCTION OF RECOMBINANT POTENCY-DETERMINING FACTORS

(75) Inventor: Lingxun Duan, San Diego, CA (US)

(73) Assignee: LD Biopharma, Inc., San Diego, CA (US)

(21) Appl. No.: 13/263,648

(22) PCT Filed: Apr. 1, 2010
(86) PCT No.: PCT/US10/29681

§ 371 Date: Nov. 16, 2011

Related U.S. Application Data
(60) Provisional application No. 61/171,819, filed on Apr. 22, 2009.
(60) Provisional application No. 61/167,843, filed on Apr. 8, 2009.

(51) Int. Cl. C12N 5/071; C12N 5/0789; C07K 2/00

(52) U.S. Cl. 435/366; 435/363; 530/300

(57) ABSTRACT

The present invention relates generally to compositions and methods for reprogramming a primate somatic cell to a higher potency level. Specifically, the invention includes compositions which comprise a recombinant polypeptide that is a potency-determining factor and methods of reprogramming a primate somatic cell to a higher potency level under conditions that allow sufficient amount of the polypeptide delivered into the primate somatic cell.

### STATEMENT OF RIGHTS TO INVENTIONS MADE UNDER FEDERALLY SPONSORED RESEARCH

Not applicable.

### TECHNICAL FIELD

The present invention relates generally to compositions and methods for reprogramming a primate somatic cell to a higher potency level. Specifically, the invention includes compositions which comprise a recombinant polypeptide that is a potency-determining factor and methods of reprogramming a primate somatic cell to a higher potency level under conditions that allow sufficient amount of the polypeptide delivered into the primate somatic cell.

### BACKGROUND ART

Human embryonic stem (ES) cells have been recognized as a valuable resource for advancing our knowledge of human development and biology, and for their great potential in regenerative medicine and drug discovery. However, previously available technologies to generate human ES dells, such as somatic cell nuclear transfer (cloning) or fusion of somatic cells with ES cells face ethical, technical and logistical barriers that impede the use of the resulting pluripotent cells in both research and therapy. Thus, the direct generation of pluripotent cells without the use of embryonic material has been deemed a more desirable approach.

A discovery toward this end was recently described, in which murine fibroblasts were reprogrammed by ectopically expressed factors known to be highly expressed in murine ES cells. Takahashi & Yamanaka, Cell 126:663-676 (2006). Specifically, transduction of a set of four genes encoding the transcription factors (TFs) Oct4, Sox2, C-Myc, and Klf4 globally reset the epigenetic and transcription network status of fibroblasts into that of pluripotent cells, designated induced pluripotent stem (iPS) cells, that were functionally indistinguishable from murine ES cells. Subsequent reports optimized this technique, demonstrating that iPS cells were indeed highly similar to ES cells when tested across a rigorous set of assays. Brambrink, et al., Cell Stem Cell 2:151-159 (2008); Statfeld, et al., Science 322:945-949 (2008); Okita, et al., Science 322:949-953 (2008); Woltjen, et al., Nature (advance online publication Mar. 1, 2009); Kaji, et al., Nature (advance online publication Mar. 1, 2009). iPS cells provide a unique opportunity to study how somatic cells de-differentiate (are reprogrammed) to an embryonic stem cell-like state, and therefore also to understand the molecular basis of cell differentiation from the pluripotent state.

Currently, iPS cell can only be generated when a set of four major stem cell specific transcription factors were transduced into various somatic cell using retroviral, lentivirial or inducible lentiviral vectors. However, it has been known that random integration of retroviral vector into host genome may alter gene function and increase the risk of carcinogenesis. Mitchell, et al., PLoS Biol 2(8):e234 (2004); Kustikova, et al., Science 308:1171-1174 (2005); Nakagawa, et al., Nat. Biotechnol. 26:101-106 (2008). Therefore, while iPS cells have enormous potential to substitute for ES cells and to generate genetically diverse and patient-specific pluripotent stem cell populations, one must overcome the risk of integrated oncogenic genes in the chromosomes of the iPS cells. Also, reported in retroviral based transduction, transduced TF genes need to be shut down in timely fashion through viral promoter DNA methylation for reprogramming endogenous TF networking regulation, post the challenge for any episomal vector based technology in industrial scale-up manufacture of iPS cells.

Therefore, there exists a need to deliver the key transcription factors into somatic cells with non-integrating approaches. Two such approaches, adenoviral delivery and transient transfection, have been successfully used in the reprogramming of mouse cells, but with much lower efficiency. Statfeld, et al., Science 322:945-949 (2008); Okita, et al., Science 322:949-953 (2008). One possible approach would be using the protein transduction technology to introduce the transcription factors into somatic cells. Because protein transduction does not involve the integration of oncogenic materials into the genome, this approach could overcome the technical challenges associated with retroviral vector-mediated transduction for iPS cell generation.

However, most peptides, or proteins, are poorly taken up by mammalian cells since they do not efficiently cross the lipid bilayer of the plasma membrane or of the endocytic vesicles. This is considered to be a major limitation for most intracellular delivery of protein either ex vivo or in vivo in basic research or clinical applications. Lebleu, B., Trends Biotechnol. 14:109-110 (1996). Proteins are currently delivered by various techniques including microinjection, electroporation, association with cationic lipids, liposome encapsidation, or receptor-mediated endocytosis. Various problems have been encountered in their use including low transfer efficiency, complex manipulation, cellular toxicity, or even immunogenicity, which would preclude their potential therapeutic applications.

All references, publications, and patent applications disclosed herein are hereby incorporated by reference in their entirety.

### SUMMARY OF THE INVENTION

Provided herein is a composition for reprogramming primate somatic cells to a higher potency level, which composition comprises a recombinant protein that is a potency-determining factor. In one embodiment, the composition comprises at least two recombinant polypeptides that are potency-determining factors. In another embodiment, the potency-determining factor is a transcription factor. In yet another embodiment, the transcription factor is selected from the group consisting of Oct4, Sox2, Klf4, Lin28, Nanog and cMyc. In a further embodiment, the composition comprises Oct4 and Sox2. In another further embodiment, the composition further comprises Klf4. In yet another further embodiment, the composition further comprises Lin28.

In one embodiment, the recombinant polypeptide is produced in E. coli and isolated from E. coli inclusion bodies. In another embodiment, the recombinant polypeptide is refolded, preferably using the pH shift technology. In yet another embodiment, the recombinant polypeptide has no post-translational modification.

In a further embodiment, the composition further comprises a compound. In another further embodiment, the composition comprises at least two compounds. In yet another further embodiment, the compounds comprise BIX-01294 and Bayk8644.

In one embodiment, the primate somatic cells are fibroblasts. In another embodiment, the primate somatic cells are keratinocytes. In yet another embodiment, the primate somatic cells are human cells.

Also provided herein is a method for reprogramming a primate somatic cell to a higher potency level, which method comprises the steps of: a) contacting the primate somatic cell with a composition for reprogramming the primate somatic cells to a higher potency level, which composition comprises a potency-determining factor polypeptide, under conditions that allow sufficient amount of the polypeptide delivered into the primate somatic cell; and b) culturing the primate somatic cell to obtain a reprogrammed cell having a higher potency level than the starting primate somatic cell. In one embodiment, the composition comprises at least two potency-determining factor polypeptides. In another embodiment, the potency-determining factor polypeptide is a recombinant polypeptide. In yet another embodiment, the potency-determining factor polypeptide is delivered into the nucleus of the primate somatic cell.

In one embodiment, the potency-determining factor polypeptide is delivered via a lipid reagent. In another embodiment, the lipid reagent is selected from the group consisting of Pro-Ject and Pulsin. In yet another embodiment, the recombinant polypeptide has a poly-arginine domain. In still another embodiment, the poly-arginine domain is derived from the HIV-1 Tat polypeptide. In a further embodiment, the recombinant polypeptide has a cell penetration domain.

In one embodiment, the primate somatic cells are cultured with the composition at a concentration of from about 0.1 μg/ml to about 40 μg/ml of the potency-determining factor polypeptide. In another embodiment, the primate somatic cells are cultured with the composition at a concentration of about 10 μg/ml of the potency-determining factor polypeptide. In yet another embodiment, the cell culturing comprises the steps of: a) growing the cells in the presence of the potency-determining factor polypeptide from about 6 hours to about 12 hours; b) rinsing the cells; and c) growing the cells in the absence of the potency-determining factor polypeptide for about 12 hours, wherein the culturing steps are repeated for at least 10 days for mouse cells and at least 21 days for human cells. In still another embodiment, the culturing steps are repeated for 14 days for mouse cells and 30 days for human cells.

Further provided herein is reprogrammed primate stem cell produced using the method for reprogramming a primate somatic cell to a higher potency level, which method comprises the steps of: a) contacting the primate somatic cell with a composition for reprogramming the primate somatic cells to a higher potency level, which composition comprises a potency-determining factor polypeptide, under conditions that allow sufficient amount of the polypeptide delivered into the primate somatic cell; and b) culturing the primate somatic cell to obtain a reprogrammed cell having a higher potency level than the starting primate somatic cell. In one embodiment, the reprogrammed primate stem cell can self-renew. In another embodiment, the reprogrammed primate stem cell can differentiate into another cell type. In yet another embodiment, the reprogrammed primate stem cell is totipotent or pluripotent. In still another embodiment, the reprogrammed primate stem cell is multipotent or unipotent.

In a further embodiment, the reprogrammed primate stem cell is an induced pluripotent stem cell. In another further embodiment, the induced pluripotent stem cell expresses an embryonic stem cell-related transcription factor. In yet another further embodiment, the embryonic stem cell-related transcription factor is selected from the group consisting of Ecat1, Esg1, Fbx15, Nanog, Eras, Dnmt31, Ecat8, Gdf3, Sox15, Dppa4, Dppa2, Fthl17, SaLL4, Oct3/4, Sox2, Rex1, Utf1, Tcl1, Dppa3, Klf4, Lin28, Ronin, Lgr5, NR6A1, ZIC3, ZFP42, FoxH1, SaLL3, Cdx2, LOC84419, EOMES, ZFX, ZFP206 and TLX.

In one embodiment, the induced pluripotent stem cell forms a teratoma when injected under the kidney capsule in nude mice. In another embodiment, the induced pluripotent stem cell shows DNA demethylation at the promoters of pluripotency genes. In yet another embodiment, the induced pluripotent stem cell is inducible to differentiate into a hematopoietic stem cell by one or more transcription factors. In still another embodiment, the transcription factor is selected from the group consisting of Runx1, Scl, Lmo-2, MLL, Tel, Bmi-1, Gfi-1 and GATA2, Hoxb4, Mesp1 and FoxA2. In a further embodiment, the induced pluripotent stem cell is inducible to differentiate into a pancreatic beta cell by one or more transcription factors. In another further embodiment, the transcription factor is selected from the group consisting of BRA, NCAD, Sox17, CER, FOXA2, HNF1B, HNF4A, PDX1, HNF6, ProX1, Sox9, NKX6-1, PTF1a, NGN3 and NKX2-2.

In one embodiment, the reprogrammed primate stem cell is a hematopoietic stem cell. In another embodiment, the hematopoietic stem cell is inducible by one or more transcription factors to differentiate into a T lymphocyte. In yet another embodiment, the transcription factor is selected from the group consisting of STATE, GATA3, STA1, T-bet, STAT4, RORC, SMAD and Foxp3. In still another embodiment, the hematopoietic stem cell is inducible to differentiate into a B lymphocyte by one or more transcription factors. In a further embodiment, the transcription factor is selected from the group consisting of E2A, EBF, LEF1, Sox4, IRF4, IRF8, Pax5, Foxp1, Ikaros and PU.1.

Still further provided herein is a primate somatic cell comprising a sufficient amount of a recombinant protein that is a potency-determining factor in the nucleus, wherein said primate somatic cell does not contain an exogenous polynucleotide encoding said protein. In one embodiment, the primate somatic cell contains at least two recombinant polypeptides that are potency-determining factors in the nucleus, but does not contain an exogenous polynucleotide encoding the recombinant polypeptides.

### BRIEF DESCRIPTION OF THE DRAWINGS

FIG. 1: Illustration of the protein-induced pluripotent stem (PiPS) cell technology. Through the recombinant protein induction in vitro, primary somatic cells can be reprogrammed into ES cell-like iPSCs. Through induction by various growth factors and key pathway-specific TFs, iPSCs can differentiate into final therapeutic cells, such as hematopoietic stem cells or red blood cells.

FIG. 2: Exemplary TFs and expression vector constructs. All five human TFs were codon optimized for E. coli using oligonucleotide-mediated PCR synthesis to obtain full-length genes. Meanwhile, the poly-arginine tag ESGGGGSPRRRRRRRRRRR was added directly to the C-terminus of each protein during gene synthesis. Finally, the gene expression cassette flanked by NdeI-XhoI sites were digested and cloned into pET41a expression vector between the NdeI-XhoI sites.

FIG. 3: Production of TF proteins using the pH shift refolding technology. All five human TF recombinant proteins were expressed in the BL21 star E. coli strain, using the auto-induction method. Inclusion bodies were purified and washed before pH shift based refolding. A final refolded protein sample with a concentration of >1 mg/ml was achieved for all five target proteins (FIG. 3A), with a purity of >90% by SDS-PAGE assay (FIG. 3B). FIG. 3B shows Oct4 and Sox2 protein samples from two refolding conditions.

FIG. 4: Characterization of human TF proteins. All purified recombinant human TF protein samples were analysis by mass spectrometry (MS) using an in-solution digestion protocol. The sample was first diluted with Rapidgest, then reduced (DTT), alkylated (IAA) and digested (trypsin) before MS analysis. A typical data is illustrated in FIG. 4A. In FIG. 4B, analyses of all five target proteins are summarized with coverage percentage for each sample. This data provides a clear identification for each purified target protein, and shows that no post-translational modification exists for any identified peptide fragment.

FIG. 5: Generating mouse iPS cells using the protein transduction method. A sample protein transduction protocol to reprogram OG2/Oct4-GFP reporter MEF cells is shown in FIG. 5A. A strategy of treating cells in 4 cycles, wherein during each cycle the fibroblasts (initially seeded at the density of 5×10⁴ cells/well in a six-well plate) were first treated with the recombinant reprogramming proteins (i.e., Oct4-11R, Sox2-11R, Klf-4-11R and cMyc-11R) at 8 μg/ml in the mESC growth media supplemented with or without 1 mM valproic acid (VPA), an HDAC inhibitor, for overnight. This was followed by changing the media to one without the recombinant reprogramming proteins or VPA, and culturing the cells for an additional 36 hours before the next cycle of treatment. After completing repeated reprogramming protein transduction, the treated cells were transferred onto irradiated MEF feeder cells and kept in mESC growth media until colonies emerged around day 30-35. In FIG. 5B, an Oct4-GFP positive colony is shown. FIG. 5C shows that when an ES-like colony was picked-up and re-seeded on feeder cells, all colonies derived from it were Oct4-GFP positive. FIG. 5D shows that the Oct4-GFP positive colonies also stained positive for alkaline phosphatase.

FIG. 6: Screening for a culture condition for recombinant protein transduction. For each target protein transduction, the protein sample was incubated with MEF cells in culture medium overnight, then the cells were washed and cultured in normal medium for 6 hours before antibody mediated immunofluorescence assay was applied. Co-staining with DAPI shows the location of nuclei.

FIG. 7: Stability study of transduced recombinant proteins. Protein transduction was performed at 8 μg/ml overnight using MEF cells, then the cells were washed and cultured in normal medium for stability tracking with immunofluorescence assay.

FIG. 8: Comparison study of poly-arginine mediated and lipid mediated protein transduction.

FIG. 9: Oct4 stability analysis by Western blot assay.

FIG. 10: Study of ES-specific gene expression in PiPS cells. Protein transduction generated mouse iPS cell colonies were analyzed for ES-specific gene expression using both immunofluorescence staining (FIG. 10A) and RT-PCR analysis (FIG. 10B).

FIG. 11: Epigenomic study of PiPS cells. Protein transduction generated mouse iPS cell colonies were analyzed for ES cell-specific epigenomic modifications such as DNA methylation at the Oct4 promoter. Two GFP-positive PiPS colonies were selected for this analysis.

FIG. 12: Amino acid sequences of OCT4: NP_{—}002692; Sox2: NP_{—}003097; KLF4: NP_{—}004226; Lin28: NP_{—}078950; and C-Myc: NP_{—}002458.

FIG. 13: Teratoma formation by PiPS cells.

FIG. 14: Generation of chimeratic mouse embryos using PiPS cells.

### DETAILED DESCRIPTION OF THE INVENTION

The present invention is intended as a solution to technical difficulties faced by retroviral vector-based iPS cell generation by using the protein transduction technology to introduce potency-determining factors into somatic cells.

### DEFINITIONS

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular form “a”, “an”, and “the” include plural references unless indicated otherwise. For example, “a” dimer includes one of more dimers.

As used herein, a “polypeptide” includes proteins, fragments of proteins, and peptides, whether isolated from natural sources, produced by recombinant techniques, or chemically synthesized. A polypeptide may have one or more modifications, such as a post-translational modification (e.g., glycosylation, etc.) or any other modification (e.g., pegylation, etc.). The polypeptide may contain one or more non-naturally-occurring amino acids (e.g., such as an amino acid with a side chain modification). Polypeptides of the invention typically comprise at least about 10 amino acids.

As used herein, the term “potency” specifies the differentiation potential (the potential to differentiate into different cell types) of a stem cell.

As used herein, the term “stem cell” refers to a cell that possess two properties: (1) the ability to self-renewal, or the ability to go through numerous cycles of cell division while maintaining the undifferentiated state, and (2) a high level of potency, or the capacity to differentiate into specialized cell types. Stem cells may have different levels of potency, which are described by different terms. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Embryonic stem (ES) cells, cells that derive from the inner cell mass (ICML) of a blastocyst, are pluripotent stem cells. Multipotent stem cells can produce only cells within one particular lineage (e.g., hematopoietic stem cells differentiate into red blood cells, white blood cells, platelets, etc.). Unipotent cells can produce only one cell type, but have the property of self-renewal which distinguishes them from non-stem cells (e.g., muscle stem cells).

As used herein, the term “induced pluripotent cells (iPS cells)” refers to cells that have been induced, either genetically or chemically, from differentiated somatic cells to cells having characteristics of higher potency cells, such as ES cells. iPS cells exhibit morphological and growth properties similar to ES cells. In addition, iPS cells express pluripotent cell-specific markers (e.g., Oct4, SSEA-3, SSEA-4, Tra-1-60, Tra-1-81, but not SSEA-1).

As used herein, the term “sufficient amount” means an amount sufficient to produce a desired effect, e.g., an amount sufficient to reprogram a somatic cell to a higher potency level.

As used herein, the term “reprogramming” refers to the process by which the potency level of primate somatic cells is increased, or the primate somatic cells are dedifferentiated, by activation or repression of cellular pathways. These pathways may be activated or repressed by nuclear transfer, cell fusion, or genetic manipulation. Reprogramming may increase the potency level of a somatic cell differently. For example, reprogramming may change the somatic cell into a pluripotent stem cell, with properties of an ES cell. Reprogramming may also change the somatic cell into a multipotent stem cell, which has the ability to differentiate into cells of a particular lineage, or a unipotent cell, which only has the ability to differentiate into a single cell type.

As used herein, a “potency-determining factor” refers to a factor, such as a protein or functional fragment thereof, that increases the potency of a somatic cell. Genes encoding proteins that are potency-determining factors include, but are not limited to, Stella, POU5F1/Oct4, Sox2, FoxD3, UTF1, Rex1, ZNF206, Sox 15, KLF4, c-Myc, Myb12, Lin28, Nanog, DPPA2, ESG1 and Otx2.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Recombinant Potency-Determining Proteins

Provided herein is a composition for reprogramming primate somatic cells to a higher potency level, which composition comprises a recombinant protein that is a potency-determining factor. A cocktail of four transcription factors, Oct4, Sox2, c-Myc and Klf4 was sufficient to mediate reprogramming across a multitude of mouse cell types, as well as rhesus monkey and human cells, to induce pluripotency. Aoi, et al., Science 321:699-702 (2008); Eminli, et al., Stem Cells 26:2467-2474 (2008); Hanna, et al., Cell 133:250-264 (2008); Kim, et al., Nature 454:646-650 (2008); Stadtfeld, et al., Curr. Biol. 18:890-894 (2008); Stadtfeld, et al., Science 322:945-949 (2008); Wernig, et al., Nat. Biotechnol. 26:916-924 (2008); Liu, et al., Cell Stem Cell 3:587-590 (2008); Park, et al., Cell 134:877-886 (2008); Takahashi, et al., Cell 131:861-872 (2007); Lowry, et al., Proc. Natl. Acad. Sci. USA 105:2883-2888 (2008). A variation on the four-factor cocktail has been used to successfully reprogram human fibroblasts: Oct4, Sox2, Nanog, and Lin28. Yu, et al., Science 318:1917-1920 (2007). The endogenous expression of certain reprogramming factors in different cell types has permitted their exclusion from the factor cocktail, such as c-Myc in fibroblasts and Sox2 and c-Myc in neural progenitor cells. Nakagawa, et al., Nat. Biotechnol. 26:101-106 (2008); Wernig, et al., Nat. Biotechnol. 26:916-924 (2008); Kim, et al., Nature 454:646-650 (2008).

In a preferred embodiment, the composition comprises at least two recombinant proteins that are potency-determining factors. In another preferred embodiment, the potency-determining factors are transcription factors. In yet another preferred embodiment, the transcription factors are selected from the group consisting of Oct4, Sox2, Klf4, Lin28, Nanog and c-Myc. In still another preferred embodiment, the transcription factors comprise Oct4 and Klf4.

Further breakthroughs in small chemical compound screening have successfully narrowed down the required potency-determining factors to Oct4 and Klf4 in MEF cells with a combination of small-molecule compounds BIX-01294 and Bayk8644. Shi, et al., Cell Stem Cell 3:568-574 (2008); Xu, et al., Nature 453:338-344 (2008). Therefore, the present invention also encompasses compositions that comprise a compound in addition to the recombinant potency-determining factor. In a preferred embodiment, the composition further comprises a compound. In another preferred embodiment, the composition comprises at least two compounds. In yet another preferred embodiment, the compounds comprise BIX-01294 and Bayk8644.

In a preferred embodiment, the recombinant protein is produced as E. coli derived inclusion bodies. In another preferred embodiment, the E. coli inclusion bodies are refolded by the pH shift technology. When a human protein is over-expressed in E. coli, more than 70% of target protein will end up in the inclusion body. Protein refolding from E. coli derived inclusion body has a high degree of uncertainty. However, the pH shift refolding technology has been applied for wtp53 protein from E. coli derived inclusion body, with a native tretramer structure, which requires Zinc metal in the refolding buffer. LaFevre-Bernt, et al., Mol. Cancer Therap. 7:1420-1429 (2008). When refolded wtp53 protein was purified and incubated with human ovarian cancer cells, it induced a specific p53 dependent cancer cell apoptosis.

In order to develop better protein refolding system, we have utilized a patented pH shift technology with various refolding buffer, which by controlling protein refolding speed and concentration, a high success rate of target protein refolding was achieved. U.S. Pat. No. 6,583,268. Other non transcription factor proteins were also successfully refolded using the pH shift technology. Lin, et al., Protein Sci. 2:1383-1390 (1993); Faro, et al., J. Biol. Chem. 274(40):28724-28729 (1999); Koelsch, et al., Biochim Biophys. Acta. 1480:117-131 (2000); Chen, et al., J. Biol. Chem. 266:11718-11725 (1991); Lin, et al., Enz. Micro. Technol. 14:696-701 (1992); Lin, et al., J. Biol. Chem. 264:4482-4489 (1989); Lin, et al., J. Biol. Chem. 267:18413-18418 (1992); Wang, et al., Biochim Biophys. Acta. 1302:224-230 (1996); Wang, et al., Science 281:1662-1665 (1998); Wang, et al., J. Mol. Biol. 295:903-914 (2000); Lin, et al., Proc. Nat'l Acad. Sci. USA 97:1456-1460 (2000); Lin, et al., FASEB J. 7:1070-1080 (1993); Rowell, et al., J. Immunol. 155: 1818-1828 (1995); Terzyan, et al., Protein Sci. 9:1783-1790 (2000); Wang & Johnsom, Anal. Biochem. 133:457-461 (1983).

In yet another preferred embodiment, the recombinant protein has no post-translational modification. Chou, et al. reported that baculoviral expression of c-Myc in sf9 cells carries an O-GlcNAc modification at Thr 58, whereas a phosphorylation at Thr 58 when expressed in human cell line. Chou, et al., J. Biol. Chem. 270:18961-18965 (1995). Another report indicated that a denatured protein may be delivered into cells more efficiently than a nature-status protein. Nagahara, et al., Nat. Med. 4:1449-1452 (1998).

The present invention may be used to reprogram a number of somatic cell types to pluripotency. For the first reprogramming attempts in both mouse and human, fibroblasts were used as the starting cell population because of technical simplicity and ready availability. A multitude of mouse cell types, including stomal cells, liver cells, pancreatic 13 cells, lymphocytes, and neural progenitor cells, as well as human keratinocytes have been reprogrammed. Aoi, et al., Science 321:699-702 (2008); Stadtfeld, et al., Curr. Biol. 18:890-894 (2008); Stadtfeld, et al., Science 322:945-949 (2008); Eminli, et al., Stem Cells 26:2467-2474 (2008); Hanna, et al., Cell 133:250-264 (2008); Kim, et al., Nature 454:646-650 (2008); Aasen, et al., Nat. Biotechnol. 26:1276-1284 (2008); Maherali, et al., Cell Stem Cell 3:340-345 (2008). In a preferred embodiment, the primate somatic cells used for reprogramming are fibroblasts. In another preferred embodiment, the primate somatic cells are keratinocytes. And in yet another preferred embodiment, the primate somatic cells are human.

### Conditions for Delivery of Recombinant Proteins

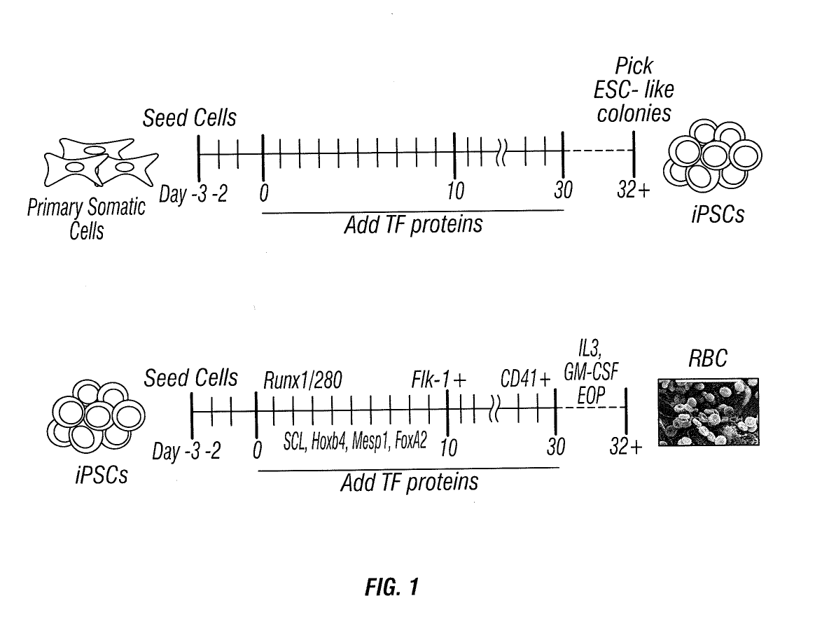

Also provided herein is a method for reprogramming a primate somatic cell to a higher potency level, which method comprises the steps of: (1) contacting the primate somatic cell with a composition for reprogramming primate somatic cells to a higher potency level, which composition comprises a recombinant protein that is a potency-determining factor, under conditions that allow sufficient amount of said recombinant protein delivered into the nuclei of said primate somatic cells; and (2) culturing the cells to obtain reprogrammed cells having a higher potency level than the primate somatic cells (FIG. 1).

Natural cell-cell movement of transcription factors in plants is a common phenomenon, has been extensively studied, and the intercellular trafficking of regulatory proteins has enraged as a novel mechanism of cell-to-cell communication in plant development. Maizel, A., Cell-cell channels (eds. Baluska, et al., 2006). Plant cell's LEAFY and APETALA1 transcription factors were demonstrated to participate in cell to cell signaling between and within different layers of the floral meristem. Sessions, et al., Science 289(5480):779-82 (2000). For mammalian cells, extracellular protein taken up by cells is also demonstrated both in vitro and in vivo for HIV-1 Tat protein, and further analysis has identified a 47-57 aa poly-arginine domain in Tat protein playing a major role for this cell membrane penetration. Gump & Dowdy, Trends Mol. Med. 13(10):443-448 (2007). Human transcription factor NeuroD has also been demonstrated for its efficient protein transduction, and final functional mapping has pin-pointed its poly-arginine and poly-lysine domain of 80-113aa. Noguchi, et al., Diabetes 54:2859-2866 (2005).

As an alternative, several peptides have been successfully used to improve the intracellular delivery of proteins. The fusogenic property of the influenza virus has been extensively studied in this context, and is currently attributed to a pH-dependent conformational change of the viral hemagglutinin leading to the exposure of its hydrophobic N-terminal region, and to the fusion of the viral and endosomal membranes. Martin, et al., Adv. Drug Deliv. Rev. 38:233-255 (1999). Also, the Tat domain fusion protein has been successfully demonstrated for intracellular delivery of several targets, such as HMGB2 and transducible p53 activation peptide. Sloots & Wels, FEBS. J. 272:4221-4236 (2005); Snyder, et al., PLoS Biol. 2(2):e36 (2004).

Therefore, in a preferred embodiment, the recombinant protein may be delivered via a lipid reagent. In another preferred embodiment, the lipid reagent is selected from the group consisting of Pro-Ject and Pulsin. In yet another preferred embodiment, the recombinant protein has a poly-arginine domain. In still another preferred embodiment, the poly-arginine domain is derived from the HIV-1 Tat protein. The HIV-1 Tat transactivation protein is efficiently taken up by cells, and concentrations as low as 1 nM in the culture media are sufficient to transactivate a reporter gene expressed from the HIV-1 promoter. Gump & Dowdy, Trends Mol. Med. 13(10):443-448 (2007); Vivés, et al., J. Biol. Chem. 272:16010-16017 (1997). The domain responsible for this translocation has been ascribed to the region centered on a basic domain of the Tat protein. A peptide extending from residues 47 to 57 allowed the internalization of conjugated proteins such as β-galactosidase or horseradish peroxidase. One to two Tat peptides/molecule of protein were sufficient to induce efficient translocation.

In a further preferred embodiment, the recombinant protein has a Cell Penetration Domain (CPD). By analyzing the human TF Mph-1, we have identified a CPD, with the peptide sequence YARVRRRGPRR, which is a part of the native peptide sequence of some human TFs, that functions to target the protein into the cell nucleus.

In a preferred embodiment, the cells are cultured at a concentration of from about 0.1 μg/ml to about 40 μg/ml of the recombinant protein. In another preferred embodiment, the cells are cultured at a concentration of about 10 μg/ml of the recombinant protein. Both mouse and human iPS cell derivation proceed under the same culture conditions used for ES cell maintenance, and it is important to ensure that the selected conditions support ES cell growth. As ES cell conditions are sufficient to obtain iPS cells from most cell types, conditions used to facilitate ES cell derivation may be used for iPS cell generation. For instance, the use of knockout serum replacement instead of fetal bovine serum greatly facilitates mouse ES cell derivation and was reported to improve the reprogramming of mouse fibroblasts. The use of knockout serum replacement provides an alternative culture condition for the reprogramming of various cell types for which standard serum is unsuitable.

Determining appropriate culture conditions for the reprogramming of non-fibroblast cell types presents a specialized case that may be tailored to satisfy the needs of both the donor cell and the arising iPS cell. Maherali & Hochedlinger, Cell Stem Cell 3:595-605 (2008). Accordingly, the reprogramming factors are typically introduced into the donor cells under their native conditions and then switched to ES cell culture conditions during the course of reprogramming, the timing of which may be experimentally determined. For instance, the reprogramming of mouse neural progenitor cells requires a switch from serum-free conditions to serum-containing ES cell conditions; if switched too early, no iPS cells are obtained. Wernig, et al., Nat. Biotechnol. 26:916-924 (2008). In some instances it is possible to employ cultures that support the growth of both the donor cell and iPS cell; for example, in the reprogramming of lymphocytes, a combination of B lineage growth factors and LIF was used, making the culture environment suitable for both hematopoietic cells and iPS cells, respectively. Hanna, et al., Cell 133:250-264 (2008).

Human iPS cell derivation also represents a unique case, as the cells are more sensitive than their mouse counterparts to the conditions under which they are grown. Maherali & Hochedlinger, Cell Stem Cell 3:595-605 (2008). For example, human iPS/ES cells display some sensitivity to doxycycline exposure, which may be accounted for when using such inducible systems. Human iPS/ES cells also exhibit poor survival when grown as single cells; accordingly, the addition of small molecules that enhance single-cell survival in established human iPS/ES cell cultures, such as the Rho-associated kinase (ROCK) inhibitor have been suggested to facilitate human iPS cell derivation, although their use is not required for successful reprogramming Maherali, et al., Cell Stem Cell 3:340-345 (2008); Watanabe, et al., Nat. Biotechnol. 25:681-686 (2007); Park, et al., Nat. Protocols 3:1180-1186 (2008).

The length of time required for cells to become independent of factor expression has been addressed using doxycycline-inducible systems. The kinetics of factor requirements has been quantified in mouse fibroblasts, which require at lease 8-12 days of factor exposure, and in human keratinocytes, which require ˜10 days. Brambrink, et al., Cell Stem Cell 2:151-159 (2008); Stadtfeld, et al., Cell Stem Cell 2:230-240 (2008); Maherali, et al., Cell Stem Cell 3:340-345 (2008). While the kinetics of reprogramming is highly influenced by the starting cell type, in all instances reprogramming requires several days to proceed. In a preferred embodiment, the cell culture comprises the steps of: (1) growing the cells in the presence of the composition from about 6 hours to about 12 hours; (2) rinsing; and (3) growing the cells in the absence of the composition for about 12 hours, wherein the culturing steps are repeated for at least 10 days for mouse cells and at least 21 days for human cells. In another preferred embodiment, the culturing steps are repeated for 14 days for mouse cells and 30 days for human cells.

### Characterization of Reprogrammed Cells

Further provided herein is an enriched population of primate cells with a higher potency level produced using the method for reprogramming primate somatic cells to a higher potency level, which method comprises the steps of: (1) contacting the primate somatic cells with a composition for reprogramming primate somatic cells to a higher potency level, which composition comprises a recombinant protein that is a potency-determining factor, under conditions that allow sufficient amount of said recombinant protein delivered into the nuclei of said primate somatic cells; and (2) culturing the cells to obtain reprogrammed cells having a higher potency level than the primate somatic cells. In a preferred embodiment, the cells can self-renew. In another preferred embodiment, the cells are totipotent or pluripotent. In still another embodiment, the cells are iPS cells.

The first generation of mouse iPS cells was obtained via selection for the ES cell-specific, but nonessential, gene Fbx15. It was later found that selection for the essential ES cell-specific genes, Nanog and Oct4, permitted the generation of iPS cells that were much more similar to ES cells. Maherali, et al., Cell Stem Cell 1:55-70 (2007); Okita, et al., Nature 448:313-317 (2007); Wernig, et al., Nature 448:318-324 (2007). With this finding also came the result that delayed onset of selection was key to generating fully reprogrammed cells, ultimately leading to the discovery that selection methods were unnecessary and actually counterproductive. Blelloch, et al., Cell Stem Cell 1, 245-247 (2007); Maherali, et al., Cell Stem Cell 1:55-70 (2007); Meissner, et al., Nat. Biotechnol. 25:1177-1181 (2007).

Additional methods to identify iPS cells have been described; these techniques become useful when one is dealing with cell types that provide a high background of non-iPS cell colonies (for example, those formed during human fibroblast reprogramming), or when ES cell expertise is lacking. Maherali & Hochedlinger, Cell Stem Cell 3:595-605 (2008). Two such methods have used ES cell-specific surface antigen expression and loss of transgene dependence as strategies to identify reprogrammed cells. For example, isolation of the Thy-1-SSEA-1+ population during the course of mouse fibroblast reprogramming greatly enriches for cells poised to become iPS cells, and live staining of cultures for the human ES cell-specific surface antigen Tra-1-81 has aided in the identification of genuine human iPS cells colonies derived from human fibroblasts. Stadtfeld, et al., Cell Stem Cell 2:230-240 (2008); Lowry, et al., Proc. Natl. Acad. Sci. USA 105:2883-2888 (2008).

Mouse iPS cells/ES cells can withstand single-cell dissociation, and newly derived colonies can be immediately subjected to enzymatic passaging, thus facilitating their quick expansion into lines. Human iPS cells/ES cells, however, survive poorly as single cells, and initial passaging of new colonies may be done mechanically; several passages (approximately five to ten) are required before the cells can be adapted to enzymatic dissociation. Lerou, et al., Nat. Protocols 3:923-933 (2008). As human iPS cells/ES cells are highly prone to differentiation, especially within the first few passages, it is important to continually remove differentiated structures to prevent them from being carried forward in the expansion. Both mouse and human iPS cell cultures can harbor initial contamination with improperly reprogrammed or differentiated cells, and subcloning may be necessary to ensure the quality of newly derived lines. Maherali, et al., Cell Stem Cell 1:55-70 (2007); Wernig, et al., Nature 448:318-324 (2007).

Several criteria have been set forth to ascertain whether a fully reprogrammed state has been achieved, which include an array of unique features associated with pluripotency, encompassing morphological, molecular, and functional attributes. Maherali & Hochedlinger, Cell Stem Cell 3:595-605 (2008). On a molecular level, iPS cells may display gene expression profiles that are indistinguishable from ES cells, which extends to the display of other associated features, including (1) protein-level expression of key pluripotency factors (e.g., Oct4, Nanog) and ES cell-specific surface antigens (e.g., SSEA-1 in mouse; SSEA-3/-4, Tra-1-60/-81 inhuman); (2) functional telomerase expression; and (3) expression of genes involved in retroviral silencing, such as de novo methyltransferases and Trim28. Lei, et al., Development 122:3195-3205 (1996); Wolf & Goff, Cell 131:46-57 (2007). iPS cells may also be epigenetically similar to ES cells, demonstrating DNA demethylation at the promoters of pluripotency genes, such as Oct4 and Nanog, X chromosome reactivation in female cells, and the presence of bivalent domains at developmental genes, consisting of overlapping histone modifications that have opposing roles. Rideout, et al., Science 293:1093-1098 (2001); Bernstein, et al., Cell 125:315-326 (2006); Maherali, et al., Cell Stem Cell 1:55-70 (2007); Wernig, et al., Nature 448:318-324 (2007). In a preferred embodiment, the iPS cells express an embryonic stem cell-related transcription factor. In another preferred embodiment, the transcription factor is selected from the group consisting of Ecat1, Esg1, Fbx15, Nanog, Eras, Dnmt31, Ecat8, Gdf3, Sox15, Dppa4, Dppa2, Fthl17, SaLL4, Oct3/4, Sox2, Rex1, Utf1, Tcl1, Dppa3, Klf4, Lin28, Ronin, Lgr5, NR6A1, ZIC3, ZFP42, FoxH1, SaLL3, Cdx2, LOC84419, EOMES, ZFX, ZFP206 and TLX. In yet another preferred embodiment, the iPS cells show DNA demethylation at the promoters of pluripotency genes.

At a functional level, iPS cells may demonstrate the ability to differentiate into lineages from all three embryonic germ layers. A hierarchy of criteria has been put forth, and in order of increasing levels of stringency, these include: (1) in vitro differentiation, (2) teratoma formation, (3) chimera contribution, (4) germline transmission, and (5) tetraploid complementation (direct generation of entirely ES cell/iPS cell-derived mice). Jaenisch & Young, Cell 132:567-582 (2008). In a preferred embodiment, the iPS cells form teratomas when 1×10⁶ cells are injected under the kidney capsule or the hind limb muscles of a 6-week-old immunocompromised SCID beige mouse.

The present invention might also be used to reprogram somatic cells to generate intermediate lineage-specific stem cells or progenitor cells or other types of multipotent or unipotent stem cells. The iPS cells generated by reprogramming somatic cells may also be induced to differentiate into multipotent or unipotent stem cells. These reprogrammed stem cells might be advantageous for differentiation and/or have a lower cancer risk because they are lineage restricted and do not form teratomas in vivo. In one embodiment, the stem cells produced using the method for reprogramming primate somatic cells to a higher potency level are multipotent or unipotent stem cells.

In a preferred embodiment, the multipotent stem cells are hematopoietic stem cells. In another preferred embodiment, the iPS cells are induced by one or more transcription factors selected from the group consisting of Runx1, Scl, Lmo-2, MLL, Tel, Bmi-1, Gfi-1 and GATA2, Hoxb4, Mesp1 and FoxA2. In yet another preferred embodiment, the iPS cells are induced to differentiate into pancreatic beta cells. In still another preferred embodiment, the iPS cells are induced by one or more transcription factors selected from the group consisting of BRA, NCAD, Sox17, CER, FOXA2, HNF1B, HNF4A, PDX1, HNF6, ProX1, Sox9, NKX6-1, PTF1a, NGN3 and NKX2-2.

The intermediate lineage-specific stem cells or progenitor cells or other types of multipotent or unipotent stem cells may further be induced to terminally differentiated cell types (FIG. 1). In one embodiment, the hematopoietic stem cells are induced to differentiate into T lymphocytes. In another embodiment, the hematopoietic stem cells are induced by one or more transcription factors selected from the group consisting of STATE, GATA3, STA 1, T-bet, STAT4, RORC, SMAD and Foxp3. In yet another embodiment, the hematopoietic stem cells are induced to differentiate into B lymphocytes. In still another embodiment, the hematopoietic stem cells are induced by one or more transcription factors selected from the group consisting of E2A, EBF, LEF1, Sox4, IRF4, IRF8, Pax5, Foxp1, Ikaros and PU.1.

Still further provided herein is a primate somatic cell comprising a sufficient amount of a recombinant protein that is a potency-determining factor in the nucleus, wherein said primate somatic cell does not contain an exogenous polynucleotide encoding said protein.

### EXAMPLES

The following examples are offered to illustrate but not to limit the invention.

### Example 1

### Human TF Gene Construction and Expression in E. coli

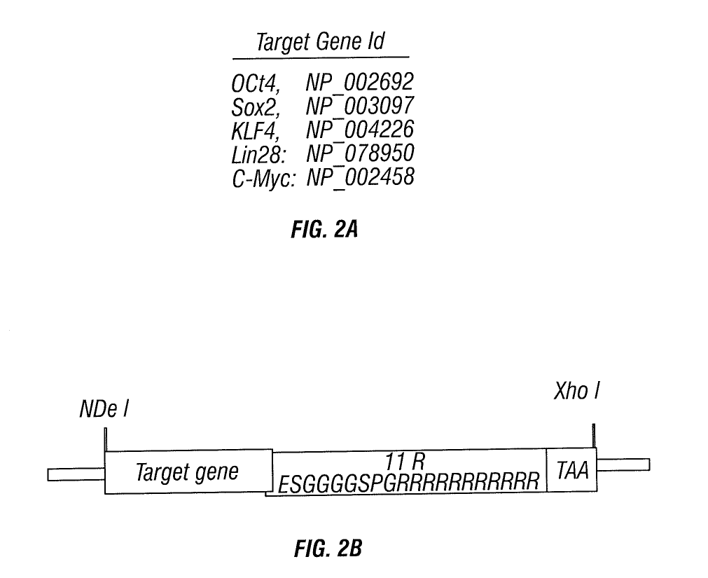

Previous reports by both Yamanaka's and Thomas' groups showed that, transcription factors, such as Oct4, Nanog, Sox2, KLF4, Myc and Lin 28, contribute to iPS cell generation. Lately, more data indicated that Nanog may be dispensable. In order to establish initial pilot test of protein based transformation assay, Oct4, Sox2, KLF4, C-Myc and Lin 28 were selected in the round of recombinant protein production for testing. The accession numbers of the sequences we used are as follows: OCT4: NP_{—}002692; Sox2: NP_{—}003097; KLF4: NP_{—}004226; Lin28: NP_{—}078950; and C-Myc: NP_{—}002458 (FIGS. 2A & 12).

### Gene Construction

In order to obtain high levels of protein expression in E. coli, all five human TF genes' codon region were optimized first, and full-length synthesized using DNA oligo based/PCR gene assembling technology. Gutman & Hatfield, Proc. Nat'l Acad. Sci. USA 86:3699-3703 (1989); Casimiro, et al., Structure 5:1407-1412 (1997). Poly-arginine tags ESGGGGSPGRRRRRRRRRRR were added to each protein's C-terminus (FIG. 2B). The final DNA fragment was flanked with NdeI and XhoI sites, and inserted into pET41 expression vector between the NdeI-XhoI sites for protein expression (FIG. 2B). Each plasmid was verified with DNA sequencing, then transformed into BL21 start competent cells for recombinant protein production using auto-induction medium overnight. Studier, Protein Expr. Purif. 41:207-234 (2005).

### Refold and Purification

The refolding was performed as described in detail in a previous p53 publication and U.S. Pat. No. 6,583,268. LaFevre-Bernt, et al., Mol. Cancer Therap. 7:1420-1429 (2008). The core refolding procedure is initiated by a “refolding screening” protocol which involves many formulation permutations of a core refolding. The purified inclusion bodies from 2 litter LB medium were dissolved in a 8 M urea buffer (8 M urea, 0.1 M Tris, 1 mM glycine, 1 mM EDTA, 10 mM (3-mercaptoethanol, 10 mM dithiothreitol (DTT), 1 mM reduced glutathione (GSH), 0.1 mM oxidized glutathione (GSSG), pH 10.5) with an OD₂₈₀=2.0. The solution was rapidly diluted into 20 volumes of 20 mM Tris plus buffer to generate a core refolding buffer of 20 mM Tris, 0.4 M urea at protein concentration around 10 ug/ml. The pH of the solution is slowly adjusted in a stepwise manner to pH 8 with 1.0 to 6.0 M HCl. In any single “refolding screening” experiment, as many as twelve parallel dilutions of 200 ml final volume were done, so that a number of other components can be included in the dilution buffer and individually tested to determine if they contribute to the success of the refolding. This is known as a refolding buffer matrix. We have tested several components which include different salts and ionic strengths, stabilizers such as arginine or glycerol, different ratios of reshuffling reagents GSH and GSSG, non-micellar dialyzable detergents, etc. The final refolded protein was then concentrated by N2 ultrafiltration, insoluble material removed by ultracentrifugation, and purified by various types of column chromatography. In the current initial refolding screening study, only a Superdex™ 200 SEC chromatography and a HPLC using a BioRad Bio-Sil SEC column were applied to assess the quantity and percentage of TF recombinant proteins in a specific refolding cocktail that can be separated from polymeric or soluble aggregated forms of the refolded protein. The refolding condition that yields the highest percent and yield of soluble protein is then utilized for large-scale refolding.

### Scale-Up Refolding and Purification

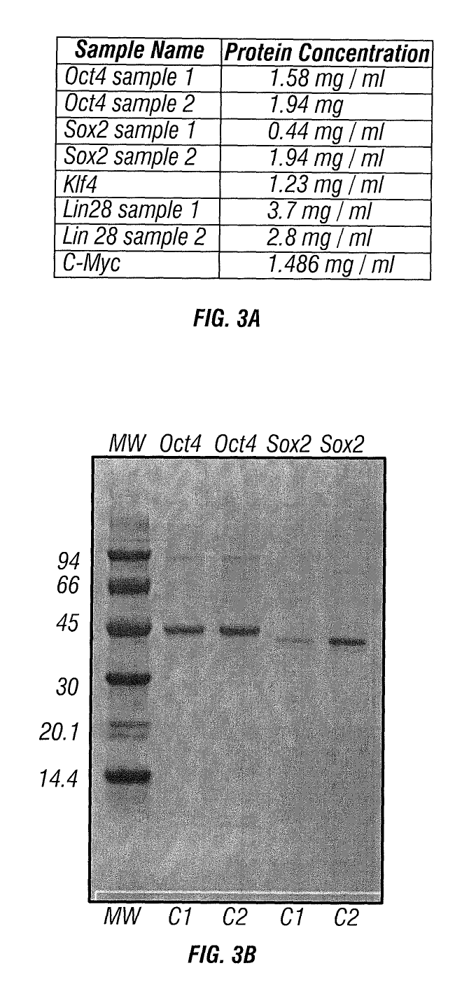

After initial optimal refolding conditions were identified for all five TF proteins in the refolding screening experiments, medium scale refolding (routinely 2×4 L containers) was carried out, again using the patented pH shift refolding methodology. Then the refolded materials were concentrated using a Millipore Pellicon ultrafiltration device. Subsequently, the protein sample was loaded on a 50 mm×100 cm Sephacryl™ S-300 SEC columns at 4° C. Purified recombinant proteins, identified by A280 absorbance and further characterized by SDS-PAGE, were combined as pooled protein sample before iPS formation assay. All protein samples demonstrated a good solubility in the buffers. See Table 1. Each sample was also tested for endotoxin, with concentration less than 100^{EU}/mg protein in all samples. Typical purity of various refolded samples is shown in FIG. 3B.

TABLE 1
Protein Solubility

| Sample Name | Protein Concentration | |
|---|---|---|
| Oct4 sample 1 | 1.85 | mg/ml |
| Oct4 sample 2 | 1.94 | mg |
| Sox2 sample 1 | 0.44 | mg/ml |
| Sox2 sample 2 | 1.94 | mg/ml |
| Klf4 | 1.23 | mg/ml |
| Lin28 Sample 1 | 3.7 | mg/ml |
| Lin 28 sample 2 | 2.8 | mg/ml |
| C-Myc | 1.486 | mg/ml |

### Initial Recombinant Protein Characterization

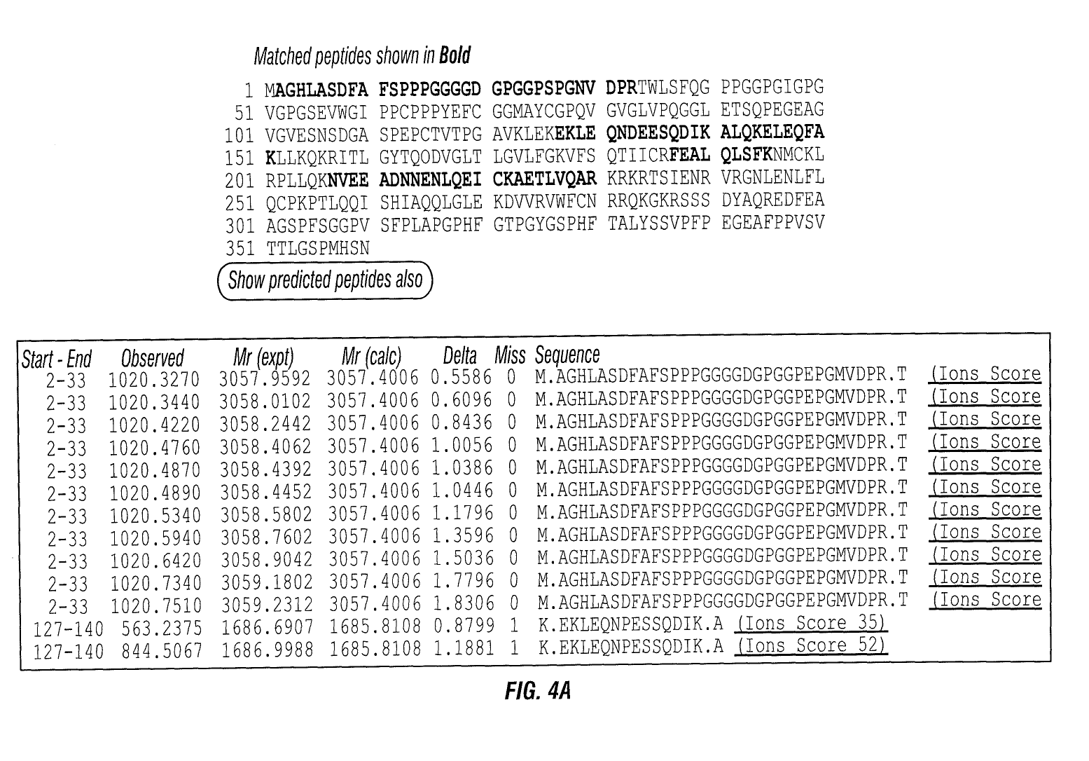

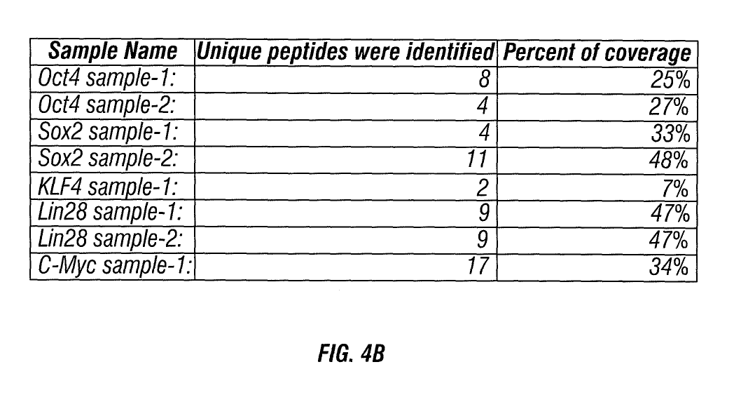

The initial TF protein refolding approach has successfully obtained several soluble proteins for each sample: Oct4 (two conditions), Sox2 (two conditions), KLF4 (one sample), Lin28 (two conditions), and c-Myc (one sample). All samples were analysis by MS using in solution digestion protocol. The sample was first diluted with Rapidgest, then reduced (DTT), alkylated (IAA) and digested (trypsin) before MS analysis. A typical data is shown in FIG. 4A.

Analysis from all identified peptide sequences indicates that there are not any protein modifications, which supports our hypothesis of “native peptide from refolded protein.” (See FIG. 4B.)

### Example 2

### Protein Delivery and iPS Assay Development

In order to quickly test the function of refold protein samples, target specific antibody based immunofluorescence test was applied initially by taking advantage of no-endogenous Oct4, Sox2 and KLF4 expression in mouse embryonic fibroblast (MEF) cells.

To further facilitate the functional screening process, the transgenic ROSA26^{+/-}/OG2^{+/-} mice were used for deriving MEF cells for testing protein transduction. ROSA26^{+/-}/OG2^{+/-} mice were derived from heterozygous Oct4-GFP (with the 18-kb Oct4 regulatory region) transgenic mice, which use GFP signal as an indicator of endogenous OCT4 gene expression pattern. Shi, et al., Cell Stem Cell 3:568-574 (2008). When OG2 MEF cells are reprogrammed into iPS cells, positive GFP signal indicates active Oct4 promoter activity.

### MEF Cell Preparation and Protein Transduction

MEFs were prepared as follows: Female mice of the OG2 strain were routinely bred at Scripps Research Institute (collaborator) animal facility. At day 12.5 of pregnancy, pregnant animals were sacrificed by cervical dislocation. Embryos were then flushed from the uteri horns and killed by decapitation. Embryos were subsequently washed in PBS and the head and the placenta were removed. The carcasses were minced, soaked in trypsin at 4° C. overnight and dissociated in a final 5 min incubation step at 37° C. Tissue was broken up by repeated pipetting and larger remaining clumps removed by letting them settle out. Cells were seeded in Invitrogen Ko-DMEM (Cat. #10829018) supplemented with 20% Knockout™ Serum Replacement (GIBCO), 2 mM L-glutamine, 1.1 mM 2-mercaptoethanol, 1 mM nonessential amino acids and cultured for testing immediately.

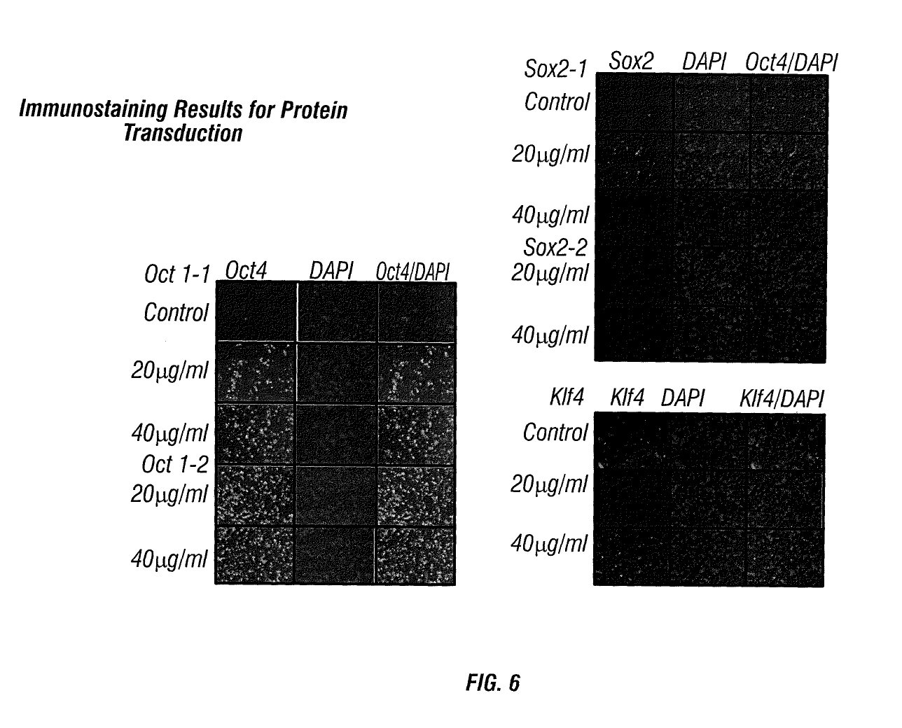

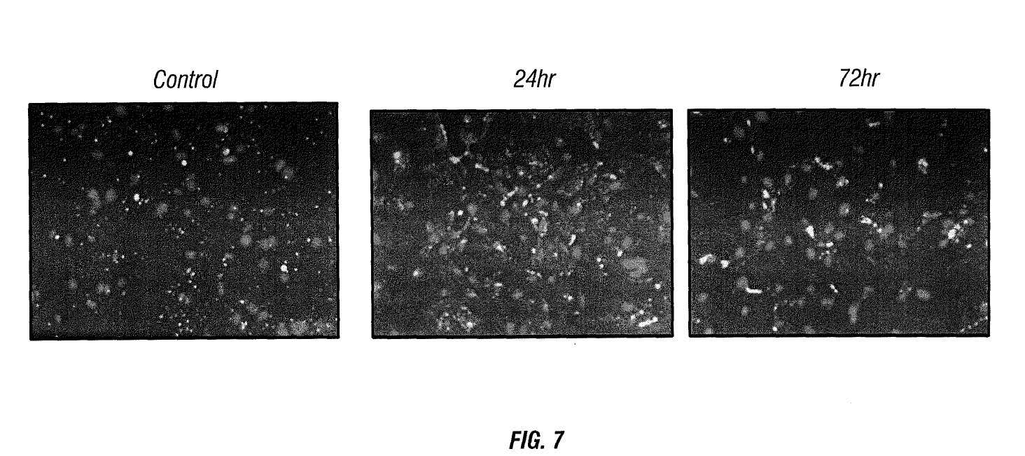

For protein based TF transduction assay, 5×10⁴ MEFs were seeded in 6-well plates, and incubated with various protein concentration which started at 1 μg/ml, 2 μg/ml, 4 μg/ml, 8 μg/ml, 20 μg/ml and 40 μg/ml for 6 hours, then the cells were fixed and immunostained by standard indirect immunocytochemistry. Anti-Oct4 (Chemicon) at 1:100, anti-Sox2 (Santa Cruz), and anti-KLF (Santa Cruz) antibodies were used. To monitor the half-life of transduced proteins in cells, after culture cells were incubated with proteins for 6 hours, cells were washed with pre-warmed fresh medium and replaced the medium without TF protein reagents, then at various time point, IF was performed to evaluate the signal level from recombinant proteins. Representative data is shown in FIGS. 6 and 7.

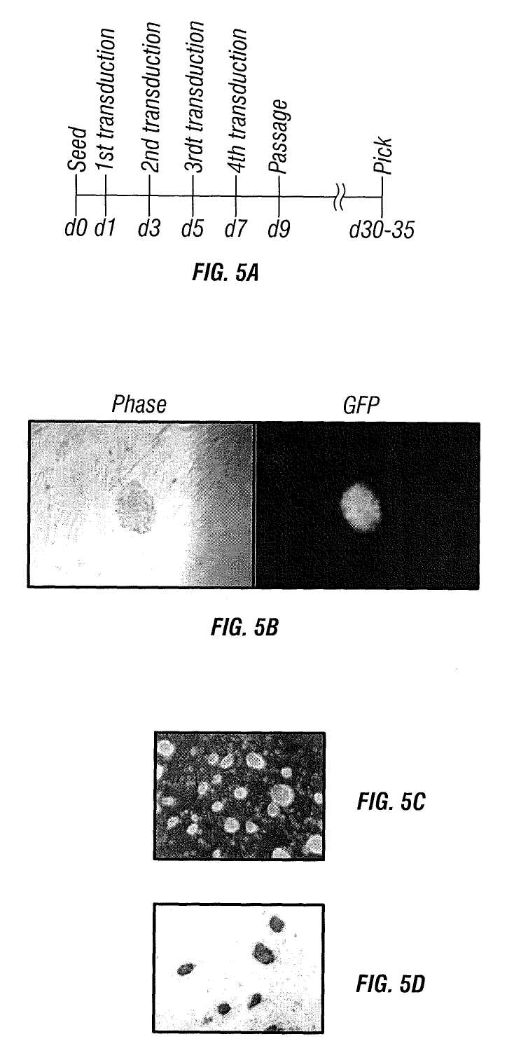

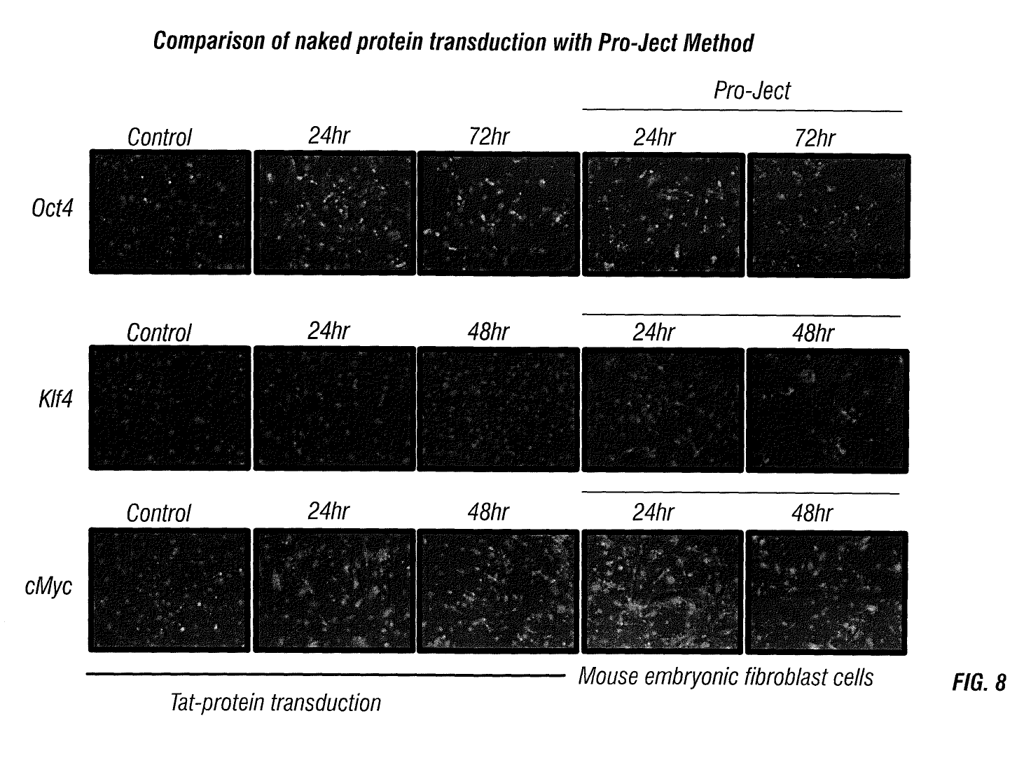

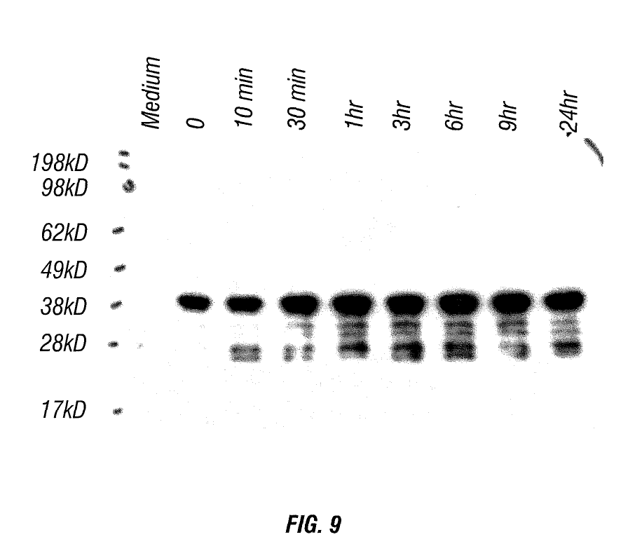

In order to compare the efficiencies between the naked protein delivery versus the lipid particle based protein delivery, such as Pro-Ject Protein Transfection Reagent (Pierce, Cat. #89850), we tested both delivery systems side by side. As data in FIG. 8 shows, both delivery systems worked with similar efficiency. Naked protein delivery at 8 μg/ml gave a good signal, but also showed the lowest cell toxicity. Meanwhile, recombinant TF protein in vitro stability study by incubating protein in culture medium with 20% FCS indicated that the recombinant Oct4 was stable for at least 24 hours at 37° C. (FIG. 9). So the following iPS transduction experiments were performed using 8 μg/ml target protein with 12 hours incubation and 36 hours protein free culture as the standard protocol. (See FIG. 5A.)

### Example 3

### Protein Transduction and iPS Cell Generation

For protein-based TF transduction treatment, 5×10⁴ MEFs were seeded in 6-well plates, and incubated with 8 μg/ml of each target protein for 12 hours in media with 1 mM valproic acid (VPA), a HDAC inhibitor. Then, cell cultures were replaced with TF protein-free media for 36 hours. This treatment was repeated for four times for mouse MEF cells. At day 9, the treated cell were trypsinized and re-seeded on MEF feeder cells using ES cell medium for culture until compact domed colonies were observed between day 21 to 30 days (after first treatment day). At this stage, GFP signal, which was driven by endogenous mouse Oct4 promoter, was clearly observed in the colonies, which indicated endogenous Oct4 gene expression 2 weeks after induction. (See FIGS. 5B & C.) Meanwhile, staining of alkaline phosphatase also provided a rapid identification of ES cell-like colony in culture. (See FIG. 5D.) Initial colony formation efficiency was estimated from 2 rounds of repeated experiments to be 1-3 colonies/5×10⁴ MEFs, similar to the control retroviral delivery system.

### Example 4

### Characterization of Protein Transduction-Induced Mouse iPS Cells

### ES Cell-Specific Gene Expression Profiling

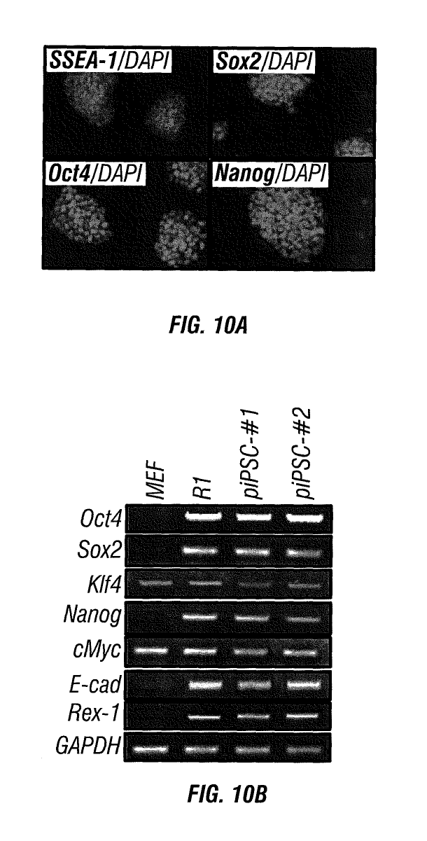

As previous retroviral derived iPS cell studies show, the fully reprogrammed iPS cells go through a global epigenomic modification changes in its genome. For example, most of the ES cell-specific genes will be fully activated in iPS cells. To characterize the ES cell-specific gene expression in iPS cells produced by the present experiments, both antibody specific immunoflorency detection and RT-PCR were performed on the iPS colonies. As data in FIGS. 10A & B shows, all commonly used ES cell-specific markers were positive for the colonies, which indicated complete epigenomic transformation in iPS cells after 2 weeks of TF protein transduction.

### OCT4 Promoter DNA Methylation Mapping

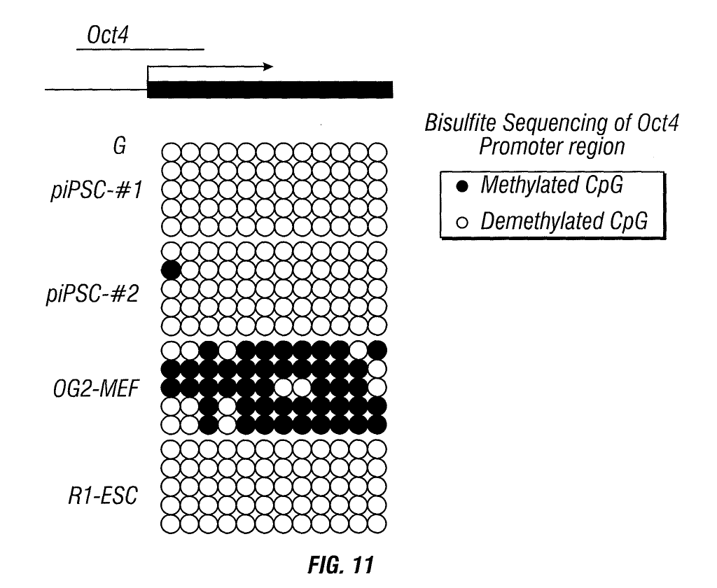

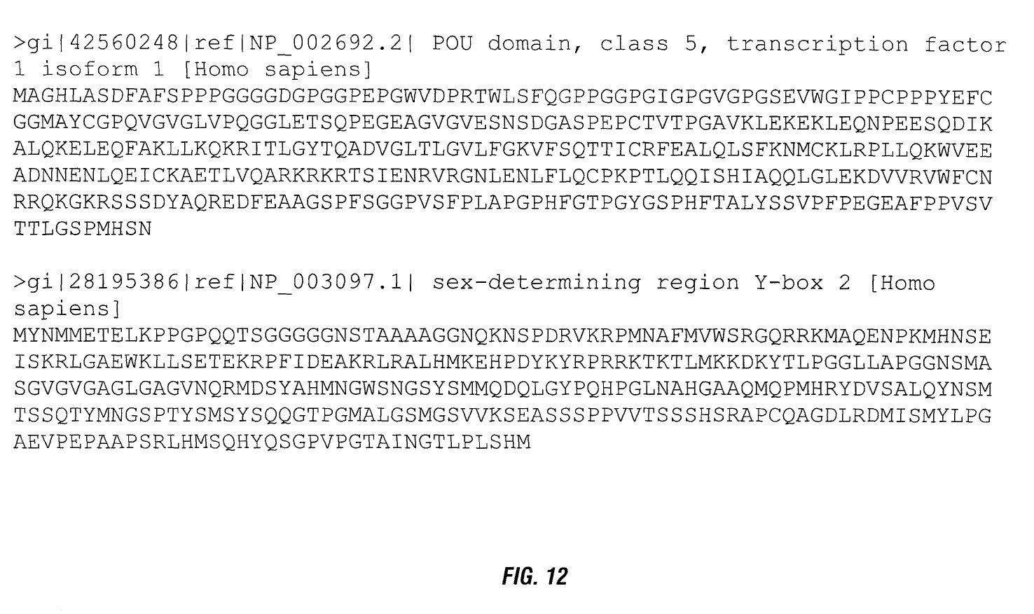

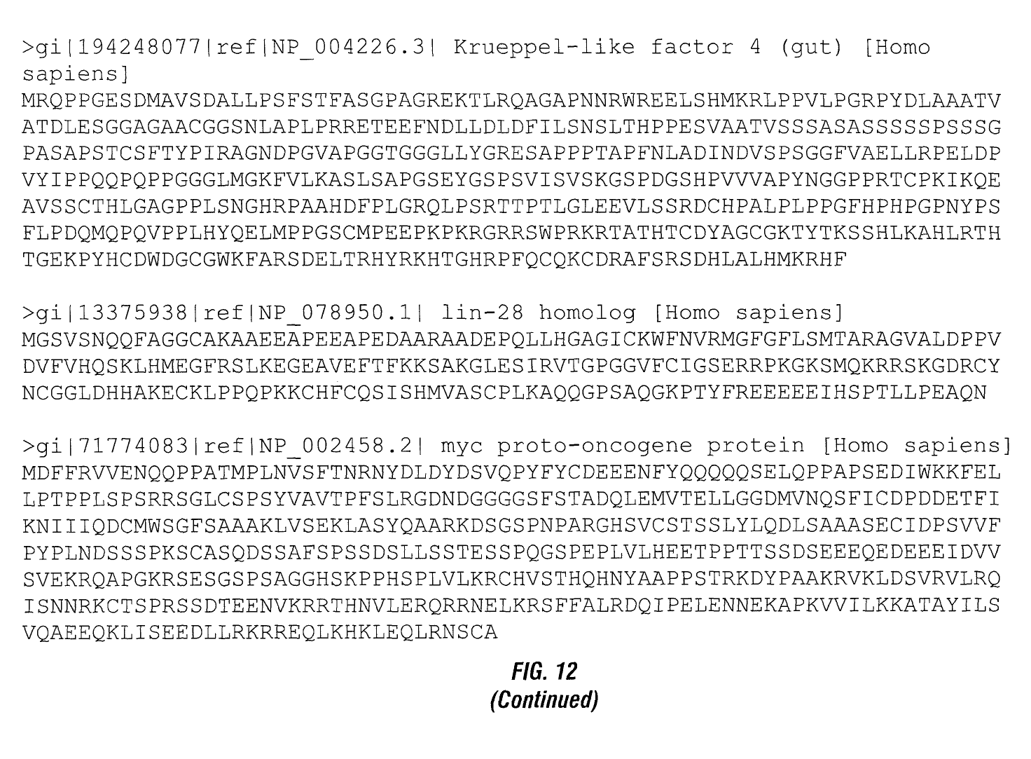

Genomic DNA from stable iPS colonies (passage 10) was isolated using the Non-organic DNA Extraction Kit (Millipore). The DNA sample was then treated for bisulfite sequencing with the EZ DNA Methylation-Gold Kit™ (Zymo Research Corp, Orange, Calif.). Primers used for promoter fragment were as previous described. Blelloch, et al., Stem Cells 24:2007-2013 (2006). The resulting fragments were cloned using the Topo TA Cloning® Kit for sequence (Invitrogen). A minimum of 2 clones were picked-up for DNA sequencing for methylation mapping at the promoter region. Data is shown in FIG. 11.

### ES Cell Surface Marker Analysis

To further monitor whether protein transduction-induced iPS cells are stable in standard ES cell culture system after 2 weeks of protein transduction treatment, all compact domed colonies were collected and re-seeded in TF protein free media for continuous culture for several weeks. At weeks 4 and 6, immunofluorescence assay was performed for more ES cell specific markers, such as Sox2, Oct4, Nanog, SSEA-4 (FIG. 10A) and alkaline phosphotase (FIG. 5D). Detection was performed without fixation in HESC media and images were taken within 1 hour after secondary antibody incubation. For early passages, iPS cells were propagated manually, whereas subsequent passage was performed with collagenase treatment as described. Shi, et al., Cell Stem Cell 3:568-574 (2008).

### Teratoma Formation Assay

PiPS Colony derived iPS cells were harvested and maintained as monolayer in chemically defined culture medium using stepwise differentiation protocol. Shi, et al., Cell Stem Cell 3:568-574 (2008). After 3-5 millions cells were injected under the kidney capsule of nude mice, all mice developed teratomas after 4-5 weeks, which were removed and then immunohistologically analyzed using specific antibodies. FIG. 13A indicated cell types from all three germ layers during development were present in the teratomas, including neural progenitor cells (Pax6+), characteristic neurons (TUJ1+), mature cardiomyocytes (CT3+), definitive endoderm cells (Sox17+), pancreatic cells (Pdx1+), and hepatic cells (albumin+) Mesoderm derived cells expressing Brachyury and mature beating cardiomyocytes expressing CT3 and MHC were also found. Images were taken with DAPI (blue) for cell nuclei staining.

FIG. 13B shows RT-PCR analysis of in vitro differentiated PiPS cell for its embryoid body (EB) development in chemically defined culture conditions.

### Chimeratic Mice Generation

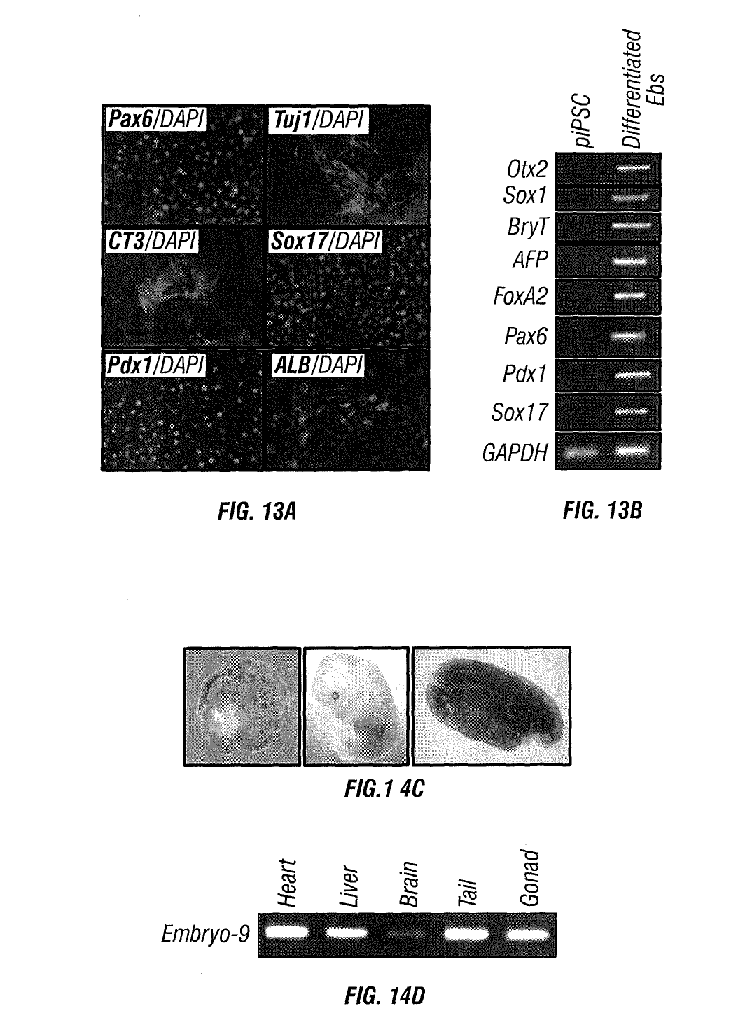

Colony derived Oct4-GFP/PiPS cells were aggregated with denuded postcompacted eight-cell stage embryos to obtain aggregate chimeras. Eight-cell embryos were flushed from females at 2.5 dpc and cultured in microdrops of KSOM medium (10% FCS) under mineral oil. Clumps of PiPS cells (10 to 20 Cells) after shot treatment of trypsin were chosen and transferred into microdrops containing zona-free eight-cell embryos. Eight-cell embryos aggregated with PiPS cells were cultured overnight at 37° C., 5% CO₂. Aggregated blastocysts that developed from eight-cell stage were transferred into one uterine horn of a 2.5 dpc pseudopregnant recipient. At day 14 of development, fetal tissue were collected from pregnant mouse and present of GFP gene in various tissues were detected using PCR amplification of genomic DNA of GFP gene, which was extracted from different layer of tissues. See FIG. 14D. Most importantly, such PiPS cells could be efficiently incorporated into the inner cell mass of blastocyst following aggregation with an eight-cell embryo and led to high-level chimerism with apparent germline contribution in vivo after the aggregated embryos were transplanted into mice, as confirmed by the presence of GFP positive cells in the inner cell mass shown in FIG. 14C.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A composition for reprogramming primate somatic cells to a higher potency level, which composition comprises a recombinant polypeptide that is a potency-determining factor.

2. The composition according to claim 1, wherein the composition comprises at least two recombinant polypeptides that are potency-determining factors.

3. The composition according to claim 1, wherein the potency-determining factor is a transcription factor.

4. The composition according to claim 3, wherein the transcription factor is selected from the group consisting of Oct4, Sox2, Klf4, Lin28, Nanog and cMyc.

5. The composition according to claim 4, which comprises Oct4 and Sox2.

6. The composition according to claim 5, which further comprises Klf4.

7. The composition according to claim 6, which further comprises Lin28.

8. The composition according to claim 1, wherein the recombinant polypeptide is produced in E. coli and isolated from E. coli inclusion bodies.

9. The composition according to claim 8, wherein the recombinant polypeptide is refolded, preferably using the pH shift technology, the pressure mediated refolding technology or the temperature shift technology.

10. The composition according to claim 9, wherein the recombinant polypeptide has no post-translational modification.

11. The composition according to claim 1, wherein the composition further comprises a compound.

12. The composition according to claim 11, wherein the composition comprises at least two compounds.

13. The composition according to claim 12, wherein the compounds comprise BIX-01294 and Bayk8644.

14. The composition according to claim 1, wherein the primate somatic cells are human fibroblasts or keratinocytes.

15-16. (canceled)

17. A method for reprogramming a primate somatic cell to a higher potency level, which method comprises the steps of:
a) contacting the primate somatic cell with a composition for reprogramming the primate somatic cells to a higher potency level, which composition comprises a potency-determining factor polypeptide, under conditions that allow sufficient amount of the polypeptide delivered into the primate somatic cell; and
b) culturing the primate somatic cell to obtain a reprogrammed cell having a higher potency level than the starting primate somatic cell.

18. (canceled)

19. The method according to claim 17, wherein the potency-determining factor polypeptide is a transcription factor.

20. The method according to claim 17, wherein the primate somatic cell is within a population of similar primate somatic cells.

21. The method according to claim 17, wherein the potency-determining factor polypeptide is a recombinant polypeptide.

22. The method according to claim 17, wherein the potency-determining factor polypeptide is delivered into the nucleus of the primate somatic cell.

23. The method according to claim 19, wherein the transcription factor is selected from the group consisting of Oct4, Sox2, Klf4, Lin28, Nanog and cMyc.

24-35. (canceled)

36. The method according to claim 17, wherein the potency-determining factor polypeptide is delivered via a lipid reagent.

37. The method according to claim 36, wherein the lipid reagent is selected from the group consisting of Pro-Ject and Pulsin.

38. The method according to claim 21, wherein the recombinant polypeptide has a poly-arginine domain.

39. The method according to claim 38, wherein the poly-arginine domain is derived from the HIV-1 Tat polypeptide.

40. The method according to claim 21, wherein the recombinant polypeptide has a cell penetration domain.

41. The method according to claim 20, wherein the primate somatic cells are cultured with the composition at a concentration of from about 0.1 μg/ml to about 40 μg/ml of the potency-determining factor polypeptide.

42. The method according to claim 41, wherein the primate somatic cells are cultured with the composition at a concentration of about 10 μg/ml of the potency-determining factor polypeptide.

43. The method according to claim 20, wherein the cell culturing comprises the steps of:
a) growing the cells in the presence of the potency-determining factor polypeptide from about 6 hours to about 12 hours;
b) rinsing the cells; and
c) growing the cells in the absence of the potency-determining factor polypeptide for about 12-36 hours,
wherein the culturing steps are repeated for at least 10 days for mouse cells and at least 21 days for human cells.

44. The method according to claim 43, wherein the culturing steps are repeated for 14 days for mouse cells and 30 days for human cells.

45. A reprogrammed primate stem cell produced using the method according to claim 17.

46. The reprogrammed primate stem cell according to claim 45, wherein the reprogrammed primate stem cell can self-renew and/or differentiate into another cell type.

47. (canceled)

48. The reprogrammed primate stem cell according to claim 45, wherein the reprogrammed primate stem cell is totipotent, pluripotent, multipotent or unipotent.

49. (canceled)

50. The reprogrammed primate stem cell according to claim 48, wherein the reprogrammed primate stem cell is an induced pluripotent stem cell which expresses an embryonic stem cell-related transcription factor.

51. (canceled)

52. The reprogrammed primate stem cell according to claim 50, wherein the embryonic stem cell-related transcription factor is selected from the group consisting of Ecat1, Esg1, Fbx15, Nanog, Eras, Dnmt31, Ecat8, Gdf3, Sox15, Dppa4, Dppa2, Fthl17, SaLL4, Oct3/4, Sox2, Rex1, Utf1, Tcl1, Dppa3, Klf4, Lin28, Ronin, Lgr5, NR6A1, ZIC3, ZFP42, FoxH1, SaLL3, Cdx2, LOC84419, EOMES, ZFX, ZFP206 and TLX.

53. The reprogrammed primate stem cell according to claim 50, wherein the induced pluripotent stem cell forms a teratoma when injected under the kidney capsule in nude mice and/or shows DNA demethylation at the promoters of pluripotency genes.

54. (canceled)

55. The reprogrammed primate stem cell according to claim 50, wherein the induced pluripotent stem cell is inducible to differentiate into a hematopoietic stem cell by one or more transcription factors selected from the group consisting of Runx1, Scl, Lmo-2, MLL, Tel, Bmi-1, Gfi-1 and GATA2, Hoxb4, Mesp1 and FoxA2.

56. (canceled)

57. The reprogrammed primate stem cell according to claim 50, wherein the induced pluripotent stem cell is inducible to differentiate into a pancreatic beta cell by one or more transcription factors selected from the group consisting of BRA, NCAD, Sox17, CER, FOXA2, HNF1B, HNF4A, PDX1, HNF6, ProX1, Sox9, NKX6-1, PTF1a, NGN3 and NKX2-2.

58. (canceled)

59. The reprogrammed primate stem cell according to claim 48, wherein the reprogrammed primate stem cell is a hematopoietic stem cell.

60. The hematopoietic stem cell according to claim 59, wherein the hematopoietic stem cell is inducible by one or more transcription factors to differentiate into a T lymphocyte, wherein the transcription factors are selected from the group consisting of STATE, GATA3, STA1, T-bet, STAT4, RORC, SMAD and Foxp3.

61. (canceled)

62. The hematopoietic stem cell according to claim 59, wherein the hematopoietic stem cell is inducible to differentiate into a B lymphocyte by one or more transcription factors selected from the group consisting of E2A, EBF, LEF1, Sox4, IRF4, IRF8, Pax5, Foxp1, Ikaros and PU.1.

63. (canceled)

64. A primate somatic cell comprising a sufficient amount of a recombinant polypeptide that is a potency-determining factor in the nucleus to reprogram the primate somatic cell to a higher potency level, wherein the primate somatic cell does not contain an exogenous polynucleotide encoding the recombinant polypeptide.

65. (canceled)

66. The primate somatic cell according to claim 64, wherein the potency-determining factor is a transcription factor selected from the group consisting of Oct4, Sox2, Klf4, Lin28, Nanog and cMyc.

67-78. (canceled)
